(19) United States
(12) Patent Application Publication
(10) Pub. No.: US 2010/0121423 A1
(43) Pub. Date: May 13, 2010

(54) Stent with Vessel Valve

(75) Inventors: Jérôme Bernhard, Zurich (CH); Rolf Jenni, Zurich (CH)

Correspondence Address: MCDONNELL BOEHNEN HULBERT & BERGHOFF LLP, 300 S. WACKER DRIVE, 32ND FLOOR, CHICAGO, IL, 60606, US

(73) Assignees: CARAG AG, Baar (CH); Universität Zürich, Zürich (CH)

(21) Appl. No.: 12/531,457

(22) PCT Filed: Mar. 6, 2008
(86) PCT No.: PCT/CH2008/000091

§ 371 Date: Sep. 15, 2009

(30) Foreign Application Priority Data
Mar. 22, 2007 (CH) 524/07

(51) Int. Cl. A61F 2/82

(52) U.S. Cl. 623/1.2; 623/1.24

(57) ABSTRACT

The invention relates to a stent that comprises a single sinus section and is preferably provided at the proximal end thereof with a native vessel valve (26). The stent comprises a first and a second hollow-cylindrical section (10, 11) having a first and a second diameter each, and a third section (12) disposed therebetween having a third maximum diameter. The third maximum diameter is greater than the first and second diameters and forms the sinus section. The stent according to the invention with the single sinus has an increased functionality. Particularly when used with a graft with a sinus and vessel valve, the functionality of the stent is secured and the wear is diminished. The stent is particularly suitable for native valves and particularly as a pulmonary valve replacement.

### TECHNICAL FIELD

The invention relates to a stent according to the preamble of claim 1, and to a stent with a vessel valve arranged therein according to the preamble of claim 4.

### PRIOR ART

Stents are supports or frameworks that are inserted into passages in the body in order to keep these passages open. They are usually brought, in compressed form, to the desired location inside a human or animal body by means of a catheter and deployed at this location. The deployment can be assisted, for example, by a balloon, by the spring action of the stent itself, or by means of shape memory. The stent usually has a lattice configuration.

Stents are best known as vascular supports for blood vessels, in particular for coronary vessels, in order to avoid renewed occlusion after the vessels have been expanded. However, stents are also used in cancer treatment where, if malignant tumors have caused a narrowing of the airways, bile ducts and esophagus, the stents maintain the patency of these passages after they have been expanded. Other areas of application are known.

U.S. Pat. No. 6,475,237 discloses a stent which is joined together from a number of individual segments, wherein the joined-together areas of the stent form nodes with a greater diameter.

US 2003/021450 discloses a stent with three hollow-cylindrical segments, of which the two outer segments each have a greater diameter than the middle segment.

US 2004/0082929 and US 2003/0236567 disclose stents with widened areas for better fixing of the stents in the vessel.

U.S. Pat. No. 4,816,029 and U.S. Pat. No. 5,469,868 disclose stents with a heart valve sewn into them.

US 2003/0009236 discloses a prosthesis with a sinus and a stent arranged therein.

US 2003/01398905 discloses a passage in the form of a cylindrical prosthesis with a widened area at one end and with a prosthetic heart valve sewn therein.

US 2001/0049553 also discloses a prosthesis for replacing an aorta passage. This prosthesis too has, at one end, a sinus in which a heart valve can be sewn in. In one illustrative embodiment, this end merges into a short cylindrical segment which, however, has a greater diameter than the segment formed integrally on the opposite side of the sinus.

U.S. Pat. No. 6,375,679 discloses a sinus made of polyurethane with a prosthetic valve.

### DISCLOSURE OF THE INVENTION

It is an object of the invention to create a stent that permits optimum flow conditions.

This object is achieved by a stent with the features of claim 1.

The stent according to the invention for maintaining the patency of a passage in the body, in particular of a blood vessel, comprises a single sinus segment and a native vessel valve. In this way it imitates the natural vessel shape and in addition, by means of this widened area, can be better placed and anchored in the vessel wall. Moreover, by virtue of the widening cross section, it optimizes the flow conditions of the body fluid, in particular of the blood, in the body passage. This reduces the strain on the valve. The native vessel valve can be of human or animal origin. For example, a native valve from a jugular vein can be used, for example a valve of a jugular vein of a horse as a pulmonary valve replacement.

The stent preferably comprises first and second hollow-cylindrical segments having first and second diameters, and, arranged between them, a third segment having a third maximum diameter, wherein the third maximum diameter is greater than the first and second diameters, and wherein it forms the sinus segment. Thus, upstream and downstream of the sinus segment, there is in each case an area of the body passage that is kept open in a predefined diameter by virtue of the stent.

The stent can be used in the form of a lattice framework or braid without any further elements. In a preferred illustrative embodiment, however, the stent is provided with a vessel valve, in particular an aortic valve or a pulmonary valve. This can be artificial, i.e. prosthetic. However, it is preferably of natural or native origin. In a preferred illustrative embodiment, a valve-supporting vessel prosthesis or a valve-supporting transplant, in each case referred to as a graft hereinbelow, is arranged in the intravascular stent.

It is a further object of the invention to create a stent with a vessel valve which permits, in addition to the optimum flow conditions, an optimum functioning of the valve.

This object is achieved by a stent with the features of claim 4.

If the vessel valve is arranged in the starting area of the third segment, as seen in the direction of flow of the body fluid, a slight liquid pressure acts on the valve in the direction against the flow. The valve can be arranged in the third segment itself or at the start thereof. It opens and closes better by virtue of the different pressure per unit of surface area on both sides, and it is therefore also subject to less wear. Upon reversal of the blood flow, the blood can flow behind the pockets or cusps of the open valve by virtue of the sinus. In this way, the valve is quickly closed and the return flow of blood minimized. Of course, the same also applies to other body fluids. In the case of a purely cylindrical stent, there is a risk that the cusps or pockets are pressed onto the stent wall and are therefore slow to close or indeed do not close at all. The result is a valve that is not tight.

The function of the valve is additionally improved if the first and second segments have the same diameter along their entire length and, as a result, the same flow conditions prevail therein.

In another advantageous embodiment of a stent according to the present invention, the area of the stent where the pockets or cusps are arranged has radially inwardly facing notches. The notches occur at the area of the circumference where two different cusps or pockets adjoin each other. In this way, a bulge is formed in the area where an individual cusp or an individual pocket is arranged along the circumference. In this way, in the cross section of the stent, a kind of rosette or cloverleaf shape is obtained in this area. This area can lie in the distal or proximal area of the third segment of the stent, that is to say, as seen in the direction of flow of the body fluid, before or in the starting area of the third segment, or in or after the end area of the third segment. In the longitudinal direction of the stent, a notch of this kind can extend along the entire sinus segment or even along the entire stent. However, it is also possible to provide the notches only at the place where the cusps or pockets are arranged or suspended. In this case, the notches have, for example, the shape of a depression in the sinus segment. Since the notch is arranged exactly where two cusps or pockets adjoin each other, the line along which the cusps or valves lie on each other for closure is shorter compared to a stent without such notches. This therefore improves the leaktightness of a closed valve. At the same time, in the case of an open valve, the through-flow surface area is reduced only inappreciably by the notches. Such notches or depressions can be obtained, for example in the case of a stent with a lattice framework as main body, by suitable shaping of the lattice framework.

Further advantageous embodiments are set forth in the dependent claims.

### BRIEF DESCRIPTION OF THE DRAWINGS

The subject matter of the invention is explained below on the basis of a preferred illustrative embodiment and with reference to the attached drawings, in which:

FIG. 1 shows a side view of a stent according to the invention;

FIG. 2 shows a longitudinal sectional view through the stent according to FIG. 1;

FIG. 3 shows a plan view of the stent according to FIG. 1, and

FIG. 4 shows a perspective view of the stent according to FIG. 1.

### WAYS OF IMPLEMENTING THE INVENTION

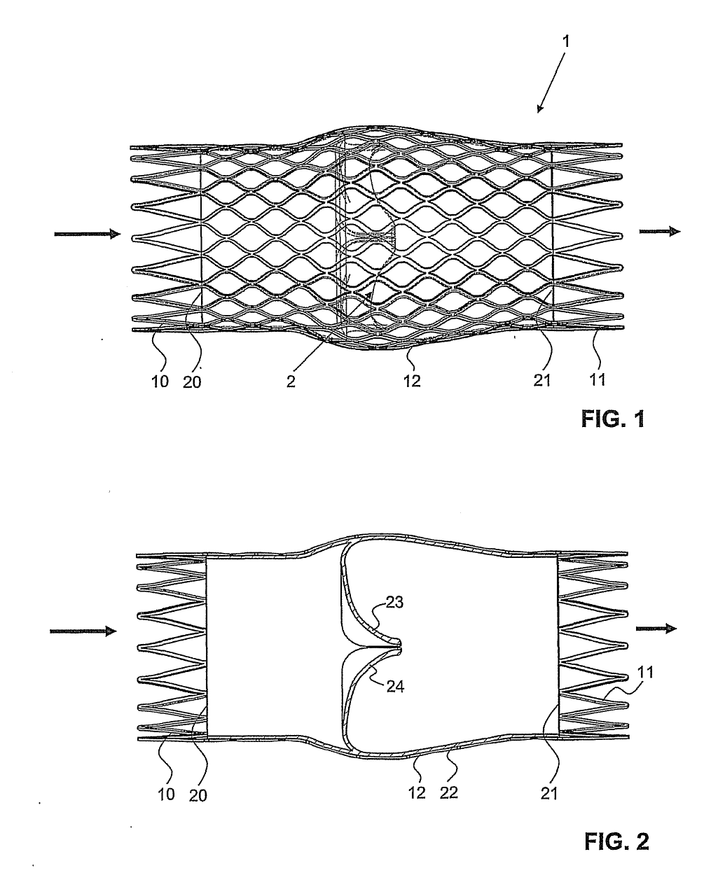

A preferred illustrative embodiment of a stent according to the invention is shown in FIG. 1 in the expanded shape of use.

The direction of flow of the body fluid, in particular of the blood, through the body passage, and therefore through the stent, is represented by arrows. In this example, therefore, the left-hand end of the stent is the proximal end, and the right-hand end is the distal end.

The stent has a main body in the form of a lattice framework 1. The lattice framework 1 can be woven or laser-cut or produced by other suitable means. The lattice framework 1 is preferably made of a metal, an alloy or a plastic. It can also be made from a biodegradable material. It is preferably made of Nitinol.

The lattice framework 1 has first and second hollow-cylindrical segments 10, 11 and, arranged between them, a third segment 12. This third segment 12 has a greater diameter than the first and second segments 10, 11. It forms the single sinus of the stent.

The first and second segments 10, 11 have an identical diameter, and this diameter remains approximately the same, preferably exactly the same, along the entire length of the segments 10, 11.

The maximum diameters of the first and second segments 10, 11 are usually 5 to 50 mm, preferably 10 to 30 mm. The maximum diameter of the third segment 12 is usually 8 to 70 mm, preferably 12 to 35 mm. Independently of the absolute values of the diameters, the ratio of the diameters of the first and/or second segment to the third segment is 1:1.1 to 1:2 and preferably 1:1.1 to 1:1.4. The example shown in the drawings has a ratio of approximately 1:1.23.

As can be seen in FIG. 1, the lattice framework 1 does not need to have the same lattice shape along its entire length. At the distal and proximal ends of the framework 1, it changes from a diamond-shaped lattice into points or triangular endpieces. Other lattice shapes are possible.

The three segments 10, 11, 12 are preferably formed from a common lattice, such that there are no seams or connection areas between the individual segments.

However, the lattice framework 1 can also be composed of individual segments 10, 11, 12 that are produced separately from one another and subsequently joined together.

The lattice framework 1 is inserted in a known manner by means of a catheter. For this purpose, it can be compressed transverse to its longitudinal direction and/or made smaller in the radial direction by being pulled longitudinally. In order to recover the expanded shape of use illustrated here, it can be of a self-expanding design, or it can be brought into this shape of use by balloon dilation or by another suitable means. The self-expansion is preferably achieved by means of elastic deformation, for example by means of a spring force, or by means of shape memory.

In its simplest configuration, the stent according to the invention can be composed only of the lattice framework 1 described above.

However, as can now be seen clearly in FIG. 2, a graft 2 with a sinus 22 and a vessel valve 26 is secured in this lattice framework 1. It is normally sewn into the lattice framework 1. However, other ways of securing it are possible, for example by adhesive bonding or clips. If the graft is sewn in, it can be secured on the lattice framework 1 by a continuous seam about the entire circumference or by individual stitches.

The graft 2 has a sinus area 22 with the valve 26 and, formed integrally thereon at both ends, tube areas 20, 21. These two tube areas, i.e. the proximal tube area 20 and the distal tube area 21, preferably lie on the inner face of the first and second segments 10, 11, respectively.

Depending on what type it is, the valve 2 has cusps or pockets 23, 24, 25. Seen in the direction of flow of the body fluid, these are arranged before or in the starting area of the sinus segment 12. In the case shown here, this means that it is arranged at the proximal end of the sinus segment 12.

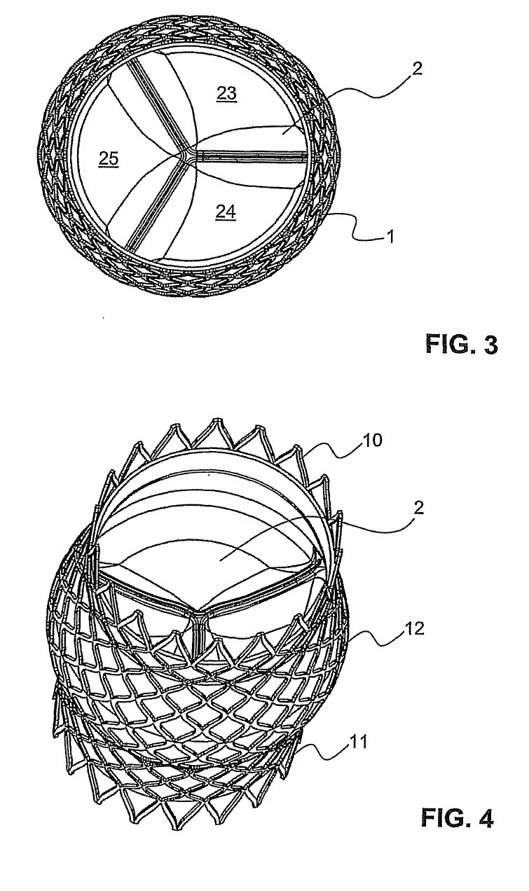

The valve 26 is preferably used as an aortic or pulmonary valve replacement. The valve itself can also be of other origin. For example, it is possible to use a native valve of a jugular vein. It is preferably a native valve of human or animal origin. In the example shown here, a valve from a jugular vein of a horse is used as a pulmonary valve replacement with the three semilunar cusps 23, 24, 25 shown clearly in FIG. 3.

Instead of a graft with sinus and valve, it is also possible for a simple vessel valve, in particular a heart valve, to be secured to the lattice framework.

The stent according to the invention, with its single sinus, provides increased functionality. Particularly when used with a graft with a sinus or another vessel valve, the functionality of the stent is secured and the wear is diminished. It is particularly suitable for native valves, in particular as a pulmonary valve replacement.

### LIST OF REFERENCE SIGNS

1 lattice framework

10 first segment (proximal)

11 second segment (distal)

12 third segment (sinus)

2 graft

20 proximal tube area

21 distal tube area

22 sinus area

23 first pocket

24 second pocket

25 third pocket

26 vessel valve

1. A stent for maintaining the patency of a passage in the body, in particular of a blood vessel, wherein the stent comprises a single sinus segment and a vessel valve, the vessel valve being a native vessel valve.

2. The stent as claimed in claim 1, wherein it comprises first and second hollow-cylindrical segments having first and second diameters respectively, and, arranged between them, a third segment having a third maximum diameter, wherein the third maximum diameter is greater than the first and second diameters, and wherein it forms the sinus segment.

3. The stent as claimed in claim 1, wherein the vessel valve is in the form of an aortic or pulmonary valve replacement.

4. The stent as claimed in claim 3, wherein the vessel valve, seen in the direction of flow of the body fluid, is arranged before or in the starting area of the third segment in this third segment.

5. The stent as claimed in claim 3, wherein the vessel valve is arranged at the proximal end of the third segment.

6. The stent as claimed in claim 3, wherein the vessel valve has a pocket.

7. The stent as claimed in claim 3, wherein the vessel valve is part of a graft which is arranged in the lattice framework.

8. The stent as claimed in claim 2, wherein the first and second diameters are of the same size, and wherein the first and second segments have the same diameter along their entire length.

9. The stent as claimed in claim 2, wherein the first and second diameters are 5 to 50 mm, in particular 10 to 30 mm, and the third diameter is 8 to 70 mm, in particular 12 to 35 mm.

10. The stent as claimed in claim 2, wherein the ratio of the first and/or second diameter to the third diameter is 1:1.1 to 1:2 and preferably 1:1.1 to 1:1.4.

11. The stent as claimed in claim 2, wherein the three segments are composed of a lattice framework.

12. The stent as claimed in claim 11, wherein the three segments are formed from a common lattice framework.

13. The stent as claimed in claim 2, wherein the three segments are self-expanding.
